(19) United States
(12) Patent Application Publication
(10) Pub. No.: US 2014/0207135 A1
(43) Pub. Date: Jul. 24, 2014

(54) ELECTROSURGICAL INSTRUMENTS, ELECTROSURGICAL DEVICE, AND ASSOCIATED METHODS

(71) Applicant: Hanno Winter, Berlin (DE)

(72) Inventor: Hanno Winter, Berlin (DE)

(73) Assignee: OLYMPUS WINTER & IBE GMBH, Hamburg (DE)

(21) Appl. No.: 14/342,214

(22) PCT Filed: Sep. 6, 2012
(86) PCT No.: PCT/EP2012/067379

§ 371 Date: Feb. 28, 2014

(30) Foreign Application Priority Data
Sep. 7, 2011 (DE) 10 2011 082 307.7

(51) Int. Cl. A61B 18/14

(52) U.S. Cl. CPC A61B 18/1445
USPC 606/40

(57) ABSTRACT

The present invention relates to an electrosurgical instrument and an electrosurgical device and related methods. According to the present invention, a water vapor which is formed during fusion is neutralized by a cooling fluid in order to prevent thermal damage of surrounding tissue.

The present invention relates to an electrosurgical instrument having a gripping surface and one at least in the area of the gripping surface arranged electrode. The present invention further relates to an electrosurgical device with an electro-surgical instrument according to the present invention. Moreover, the present invention relates to a method for the operation of an electrosurgical device as well as to a method for tissue fusion.

Electrosurgical instruments are e.g. used for transecting, coagulating and thermally sealing vasculature. For this purpose, the impedance-controlled bipolar high-frequency technology was developed, which provides a low-cost and in the field of surgery established method. Depending on temperature, time and pres-sure, it is principally possible to fuse also other kinds of tissue, like e.g. intestinal wall, urethra or the skin, and thereby to close wounds. For this purpose, a thermally induced transformation process of proteins being present in the human body, which is also designated as denaturation, can be used. For a successful wound closure by heating of the tissue, a possible thermal damage of the tissue cells, which may occur due to overheating in the area of the edge of a fusion suture, shall be as low as possible and locally confined.

If the biological tissue is heated during the fusion process above 100° C., the cell fluid evaporates and the tissue dehydrates. A water vapor which is formed in the tissue and which discharges from the tissue contributes through condensation on the relatively cool surface of the surrounding tissue to thermal damage. For the prevention of such damage, already various approaches have been made.

From document U.S. Pat. No. 7,789,883 B2 a device for thermo fusion in known in which, through special configuration of the electrodes or through channels in the edge area of an electrode, a lateral spreading of the water vapor shall be prevented. In this document, also a cooling unit in the edge area of the electrode is described.

Document DE 607 38 220 T2 describes an electrode with bores, through which water vapor can be withdrawn by suction during heating.

Document U.S. Pat. No. 7,815,641 B2 discloses an electrosurgical instrument which has, besides electrodes, at least one cooling unit for causing a temperature gradient between the electrodes and the cooling unit.

Document U.S. Pat. No. 5,647,871 A1 discloses an electrosurgical instrument which has an electrode with cooling channels arranged therein. By feeding a cooling fluid, the electrode can be cooled.

It would be desirable to prevent thermal damage caused by the formed water vapor in an alternative, particularly improved manner.

According to the present invention, for this purpose, an electrosurgical instrument and an electrosurgical device according to the independent method claims are provided. Further, a method for operating an electrosurgical device and a method for tissue fusion according to the independent method claims are provided. Advantageous embodiments can e.g. be derived from the dependent claims.

According to a first aspect, the invention relates to an electrosurgical instrument having a gripping surface and one at least in the area of the gripping surface arranged electrode. Further, in the electrosurgical instrument, outside of the gripping surface adjacent to it, a fluid outlet is arranged, which is connected to the fluid channel for supplying a cooling fluid.

The cooling fluid represents a defined drain for the water vapor and the energy stored therein. When the water vapor arrives at the cooling fluid, it condensates in the cooling fluid, whereupon the heat of condensation formed thereby heats the cooling fluid. Subsequently, the condensed water will cool down to the temperature of the cooling fluid, whereby again energy is released which is absorbed from the cooling fluid. If cooling fluid is supplied in a sufficient dose, the cooling fluid nevertheless does not evaporate but dissipates the heat. Thereby it is pre-vented that the energy, which is formed during condensation and cooling of water vapor, heats and thus damages the tissue outside of the desired area.

The invention comprehends the finding that the effect of the methods known from the prior art for reducing thermal damage of tissue surrounding the fusion suture is only limited. So, the groove surrounding the electrodes indeed prevents a damage beyond the width of the groove, however, the groove must have a mini-mum width in order not to be clogged by the tissue fluid-cell-conglomerate which is formed during the fusion process. Therefore, the damage can only be reduced by this method insofar as the groove is wide enough. In bores for extracting water vapor being known from the prior art, the danger of clogging of the bores also occurs.

Further, the present invention comprehends the finding that water vapor can be dissipated best by flushing the electrodes or the tissue butting against the electrodes. A heat rejection through a cold, electrically non-conductive fluid stream is significantly more effective than dissipating or withdrawing water vapor by suction.

In a preferred embodiment, at the electrosurgical instrument, outside the gripping surface and adjacent to it, a suction opening for withdrawing the cooling fluid by suction is arranged. Thereby, the cooling fluid which has discharged from the fluid outlet and which was heated by the condensed water vapor can again be withdrawn by suction. An accumulation of cooling fluid at the electrosurgical instrument or in an organ to be treated is thus prevented. Such suction opening also allows a continuous stream of cooling fluid along the electrode. Thereby, the stream can be adjusted to the required cooling capacity.

The gripping surface of the electrosurgical instrument gets during use of the instrument into contact with tissue. The electrode arranged in this area has preferably a surface of conductive material, e.g. of a metal, like stainless steel or aluminium. The electrode is typically connected to a high-frequency (HF) genera-tor by means of a connecting wire, which can apply a high-frequency voltage to the electrode. Thus, during appropriate contact of the electrode with a tissue, to which also a counter electrode is applied, also a HF current can flow through the tissue.

The fluid outlet can be a simple opening in a body of the electrosurgical instrument. Typically, the opening thereby directs towards the outer face, i.e. to the adjacencies of the electrosurgical instrument. The fluid channel can be configured by a tube or a duct in the inner part of the electrosurgical instrument. Thereby, a particularly simple embodiment is facilitated.

Instead of using only one fluid outlet, however, also several fluid outlets can be used. Thereby, a distribution of the cooling fluid over a specific area or the supply to several areas can be achieved. A fluid outlet can also be structured specially in order to direct the cooling fluid during discharge to a certain direction.

Instead of a tube or a duct, the electrosurgical instrument can also at least partial-ly be configured as a hollow body, into which the cooling fluid is fed and in which at least one fluid outlet is configured. Thereby, also additional cooling of the instrument can be achieved.

The arrangement of the fluid outlet as close as possible to the electrode is preferred. Thereby, a thermally damaged area around the electrodes can be kept as small as possible or be avoided, respectively. Preferably, between the cooling fluid and the electrode, a good thermal isolation, e.g. in the form of an insulating layer, is provided. This prevents that through excessive cooling of the electrodes themselves too much thermal energy is dissipated, so that a quick heating-up of the fusion suture is prevented. It is further advantageous if this thermal insulation layer is also electrically insulating in order to prevent a lateral current flow over the cooling fluid, which is preferably electrically non-conductive, since due to washing out of electrolytes from the tissue the cooling fluid can lose its electrically insulating property in the area of the electrode.

According to a preferred embodiment, the electrosurgical instrument has two gripping surfaces facing each other and being movable towards each other. The gripping surfaces are thereby the faces most closely oppositely facing each other. It is to be understood that, in such case, at least one electrode is arranged in the area of each gripping surface. Hence, such instrument has in total two electrodes of different polarity, which can be used for passing electrical current through the tissue to be treated.

If the electrosurgical instrument has two gripping surfaces, regarding the fluid outlet basically two embodiments are possible. At the one hand, it is possible that only adjacent to a gripping surface a fluid outlet or also a plurality of fluid outlets is provided, i.e. no fluid outlet is arranged at the other electrode. On the other hand, however, it is also possible that adjacent to the both gripping surfaces, respectively, a fluid outlet or also several fluid outlets are provided, so that cooling fluid can discharge adjacent to both electrodes. In this case, water vapor can not only be absorbed or cooled by the cooling fluid at one electrode, but at both electrodes.

According to a preferred embodiment of the electrosurgical instrument comprising two gripping surfaces, the electrosurgical instrument has two branches being jointed with each other and being movable towards each other, wherein the grip-ping surface is formed by a surface facing the respective other branch. A typical example of such embodiment is a plier-like configuration, in which the branches are formed by components of the plier-like instrument. Thereby, the electrosurgical instrument can also become an electrosurgical gripping instrument. If the branches can be moved towards each other close enough that intermediary positioned tissue can be gripped, i.e. be contacted on both sides and held in position with a respective force, the electrosurgical instrument can thus be fixed to the tissue.

According to a second aspect, the invention relates to an electrosurgical device comprising an electrosurgical instrument according to the first aspect and a fluid pump. The fluid pump is connected to the fluid channel for supplying a cooling fluid.

The electrosurgical device further comprises a generator for generating a coagulation current, which is electrically connected to the electrode arranged in the area of the gripping surface of the electrosurgical instrument. Further, the fluid pump and the generator are connected to a controller which coordinates the operation of the fluid pump and the operation of the generator with each other. The coordination of fluid pump and generator can take place such that the heating capacity effected by the generator and the cooling capacity generated by the fluid pump are harmonized.

The electrosurgical device according to the second aspect takes advantage of the already with regard to the electrosurgical instrument according to the first aspect of the present invention described advantages. The possible embodiments and modifications referred to are also feasible accordingly for use of such electrosurgical instrument within the scope of an electrosurgical device according to the second aspect of the present invention.

The electrosurgical device according to the second aspect of the invention allows an electrosurgical treatment of tissue, whereby a thermal damage of the tissue outside of the coagulation area is prevented by means of the cooling fluid supplied via the fluid pump.

The fluid pump could be any pump being suitable for pumping liquids or respective cooling fluids, e.g. a piston pump, a centrifugal pump or a diaphragm pump, preferably a peristaltic pump. The generator is preferably a HF generator, which is known from the prior art for the use with electrosurgical instruments. Typically, the generator provides a HF power which suffices for coagulating, fusing or otherwise treating of tissue. The generator can be connected either only to one electrode of the electrosurgical instrument used for the electrosurgical device and additionally to a back electrode, which is applied at the body of the patient to be treated. If the electrosurgical instrument used for the electrosurgical device has at least two electrodes, the generator can also be connected to two electrodes of said electrosurgical instrument. It is particularly advantageous, if the electrosurgical instrument is an electrosurgical gripping instrument and the generator is connected to two oppositely facing bipolar electrodes at branches being movable to each other and jointed with each other. In this case, the current flow through the tissue can be locally limited.

The controller coordinates the operation of the fluid pump and the operation of the generator. This can e.g. comprehended such that the controller controls the operation of the fluid pump in such that a sufficient amount of cooling fluid is permanently supplied in order to cause condensation of the water vapor formed due to the coagulation effect caused by the generator. By these means, a thermal damage of the tissue is avoided. Such controller can e.g. be connected to one or several temperature sensors for monitoring the temperatures of the supplied and/or dissipated and/or in the body present fluid and/or of the fused tissue. Thereby, the controller can recognize when the supplied fluid amount is no longer sufficient to absorb and to dissipate the thermal energy being present due to the water vapor.

According to a preferred embodiment, the controller is configured to control the generator such that it generates pulsating HF current. Thereby, the cooling effect is significantly improved, as will be specified below.

A pulsing HF current, in combination with a convective cooling which is provided to the tissue to be coagulated by means of the cooling fluid, leads to a significant reduction of the volume of the water vapor which is produced at one time. Through permanent convective 5 cooling, the tissue can cool down again after a short as possible stress caused by heat. Additionally, the surrounding tissue is cooled by a cold cooling fluid. Through this, the temperature of the tissue does not rise so extremely per pulse. For supporting this effect, it is advantageous if the supply of the cooling fluid takes place before applying the coagulation circuit.

In a pulsed application, it is preferred that, in a pulse as short as possible, only a small amount of the tissue fluid is evaporated in the area to be coagulated. In other words, the tissue fluid is not evaporated at once, but only in small doses. These doses have significantly less thermal energy than it would be if the entire water would be evaporated at once. The temperature of the cooling fluid and of the surrounding tissue does not rise as extremely as it would be the case with a higher amount. Preferably, the pulses release exactly as much energy to the tissue as is sufficient to allow the temperature to rise in the tissue only for a short time to the (for fusion sufficient) for evaporation necessary temperature of ebullition.

Since also the effects of heat conduction like convection cause tissue damage in the edge areas of the electrodes, it is desired that the temperature of ebullition is reached as fast as possible. Thus, the edge of temperature rise should be as steep as possible. However, since the tissue resistance rises significantly and quickly when the desired temperature in the tissue is reached, this high energy can be maintained only very shortly, because otherwise, due to the quick rise of the output voltage, electric arcs between the electrodes might occur. Thereby, the tissue between the electrodes could be destroyed and carbonized.

In order to configure the controller of a pulsed release of HF current as efficient as possible, various control techniques can be applied.

A possible control algorithm is designated as resistance-controlled and voltage-controlled application. Thereby, it is initially tried to maintain the released energy constant by adjusting the output voltage provided by the generator. Thus, the voltage to be applied depends on the tissue resistance. During the transition between liquid and gaseous phase of the tissue fluid, a quick rise of this tissue resistance occurs, whereby also the output voltage rises accordingly. In order to only release as much energy per pulse as necessary that indeed tissue fluid evaporates but the voltage does not rise too much, a resistance-controlled pulse length is advantageous. The pulse is automatically terminated if a preset resistance barrier is exceeded. Since with the dehydration of the tissue the tissue resistance increases from pulse to pulse and during fusion typically a maximum dehydration degree shall be reached, it is advantageous to increase the resistance barrier gradually with each pulse. Hereby, also the pulse length increases gradually with each pulse. The level of the switch-off threshold depends on very different parameters and can be configured individually depending on the application. It is advantageous to realize the interval times between pulses by means of a time control in order to ensure that the pause length suffices to again cool the surrounding tissue.

In such control, it is further advantageous to limit the length of the pulses by a resistance threshold. Due to dehydration of the tissue, the resistance rises with each pulse, which can be measured both during the pulses and also during the pulse intervals, i.e. between the pulses. The level of resistance during the pulses due to evaporation of tissue fluid is only of short-term nature, because a part of the vapor is not pressed out of the heated volume and immediately condenses again in the tissue. In contrast thereto, however the resistance during pulse intervals represents a degree of a prolonged persisting dehydration condition. Since in tissue fusion particularly the prolonged portion is of importance, it is advantageous to terminate the application after having reached a resistance threshold value for the resistance in the pulse pauses.

A temperature-controlled and voltage-controlled application represents an alter-native to the resistance-controlled and voltage-controlled application. Thereby, the achievement of a temperature threshold is detected by continuous measuring of the tissue temperature by means of at least one in an electrode integrated heat sensor. If the tissue temperature reaches a predetermined temperature limit, like e.g. 100° C., the pulse is automatically terminated. If the temperature drops again below a lower temperature threshold, which e.g. can be at 30° C., the pulse is started again. Due to a by each pulse increasing tissue resistance, the pulse power will decline because of a voltage threshold. Through this, also the length of time increases which is necessary to heat the tissue to the upper temperature limit. As a consequence, the pulses become typically longer over the time.

The described temperature-controlled and voltage-controlled application has the advantage that the lengths of pulses and pauses—and thus also the energy release—of the generator automatically adjust to the type of tissue and other parameters, which e.g. depend on the used instrument. Thereby, also in different applications, the same temperature of the tissue between the electrodes can be generated. Also in this case it is however advantageous to limit the total duration of the application by a resistance threshold value. This can be effectuated as described with respect to a resistance-controlled and voltage-controlled application.

According to a preferred embodiment, the electrosurgical device according to the second aspect of the invention further comprises a suction pump by which the cooling fluid can be withdrawn by suction. This allows the discharge of cooling fluid not only in the vicinity of the electrode, i.e. typically at the tissue and hence in the body of a patient, but also to withdraw it by suction from this area. An accumulation and uncontrolled distribution of cooling fluid in the body of the patient can thereby be avoided.

The suction pump can be configured in a variety of common modes, e.g. in the form of a piston pump, a centrifugal pump or a diaphragm pump. Preferred are peristaltic pumps.

At the one hand, it is possible that the suction pump again reintroduces the cool-ing fluid withdrawn by suction into a circulation and leads it back again via the fluid pump to the fluid outlet. In other words, in such embodiment, the cooling fluid withdrawn by suction can be reused. Preferably, in such case, the cooling fluid withdrawn by suction is purified before recirculation, which can be effected e.g. by means of a filter, and/or cooled, which can be effected e.g. by means of a cooling device. It is to be comprehended that, in such case, the fluid pump can at the same time operate as a suction pump, e.g. that indeed only one pump is provided in the cycle.

Alternatively, the cooling fluid withdrawn by suction can be fed into a storage system or a disposal system, like e.g. a tank or a discharge pipe. In such case, it will not be re-used.

For withdrawing cooling fluid by suction, a separate hose with a suction port can be provided which can be introduced into the patient's body independent of the electrosurgical instrument. Thereby, the withdrawal of cooling fluid by suction can take place in a flexible manner, i.e. the hose can be positioned exactly at the position of the body, at which the cooling fluid shall be withdrawn.

Alternatively, however, the electrosurgical instrument can have a suction port for withdrawing cooling fluid by suction outside the gripping surface adjacent to it. This suction port is then connected to the suction pump. This allows that the suction pump withdraws the cooling fluid via the suction port, which has a defined position at the electrode. Thereby, a predetermined fluid channel along the electrode can be provided.

According to a preferred embodiment, the electrosurgical device is configured such that the fluid pump supplies the cooling fluid during operation at a temperature of 1° C. to 6° C. and preferably between 1° C. and 3° C. In practice, this value range proved to be particularly advantageous. Such temperature can e.g. be reached in that the electrosurgical device further comprises a cooling unit, which can have e.g. a Peltier element or a compressor-powered cooling unit. However, for heat rejection, the cooling unit can be also connected to an external cooling circuit, which is e.g. installed in the building. Alternatively, the supply of the fluid at a temperature of 1° C. to 6° C. respectively 3° C. can also be achieved in that the cooling fluid is already provided at a respective temperature. Fort this purpose, vessels with the cooling fluid can e.g. be stored in a refrigerator and be taken out only shortly before use.

According to a third aspect, the present invention relates to a method for the operation of an electrosurgical device. The method comprises the following steps:
Applying AC voltage to at least one electrode of a gripping surface of an electrosurgical instrument,
supplying a cooling fluid in direct proximity of the electrode in coordination with the application of the AC voltage.

The method according to the third aspect of the invention can be used advantageously if tissue shall be fused. Through coordinated supply of cooling fluid in direct proximity of the electrode, thermal damage of the tissue is prevented.

The method according to the third aspect of the invention is preferably performed with an electrosurgical device according to the second aspect of the invention. It can also be performed only with an electrosurgical instrument according to the first aspect of the invention. The variants of embodiments and advantages de-scribed there also apply for the method according to the third aspect of the pre-sent invention. Particularly, the cooling fluid preferably is supplied in such dose that the water vapor can, to a large extent, substantially condense completely and that the heating of the cooling fluid taking place thereby does not exceed an admissible value. Further, it is also preferred that the cooling fluid is discharged at a temperature of 1° C. to 6° C. respectively 1° C. to 3° C. and that the AC voltage is supplied in pulsed mode, as already described in detail above.

However, the process can also be performed without the use of an electrosurgical device according to the second aspect of the present invention. It can particularly also be performed in such that a common electrosurgical instrument is used and, independent thereof, a flushing of the tissue section to be coagulated along fluid channels is provided. This can e.g. be effectuated such that cooling fluid is pumped to the proximity of the tissue section to be coagulated by means of a pump and a hose, and that it is again withdrawn by suction by means of a further pump and a further hose.

Particularly preferred, the fluid stream is consistent, which allows a consistent heat rejection.

Preferably, a non-conductive fluid is used as cooling fluid. Thereby, a possible short circuit, which might occur during penetration of cooling fluid between the electrodes, is prevented. For this, e.g. an electrolyte-free solution can be used. Such is currently distributed under the trade name Purisole® of Fresenius Kabi AG, Bad Homburg.

According to a fourth aspect, the invention relates to a method for tissue fusion, comprising the following process steps:
Pressing the tissue sections to be fused against each other in a fusion zone,
heating the tissue sections to be fused in the fusion zone, and
cooling the tissue by supplying a cooling fluid adjacent to the fusion zone.

In the method according to the fourth aspect of the invention, two tissue sections can be fused with each other in one fusion phase. This means that they are subsequently permanently connected with each other. The method according to the fourth aspect of the invention is preferably performed with an electrosurgical device according to the second aspect of the invention or with an electrosurgical instrument according to the first aspect of the invention. The variants and ad-vantages described therein are also applicable to the process steps according to the fourth aspect of the invention. Particularly, the method according to the fourth aspect of the invention facilitates a prevention of thermal damage outside the fusion zone, since the tissue is cooled by the supplied cooling fluid.

The step of heating comprises according to one embodiment the feed of a coagulation current into tissue sections to be coagulated. According to another, however not necessarily alternative embodiment, the step of heating can also comprise heating of tissue sections to be coagulated by means of at least one heating unit. Both embodiments can also be combined, i.e. the tissue can be heated either simultaneously or also alternating with a coagulation current and a heating unit. Heating by means of a heating unit is particularly appropriate if the resistance due to dehydration of the tissue is already highly increased.

Further advantages and embodiments of the present invention will become obvious to the skilled person when studying the following embodiments which are described with respect to the attached figures.

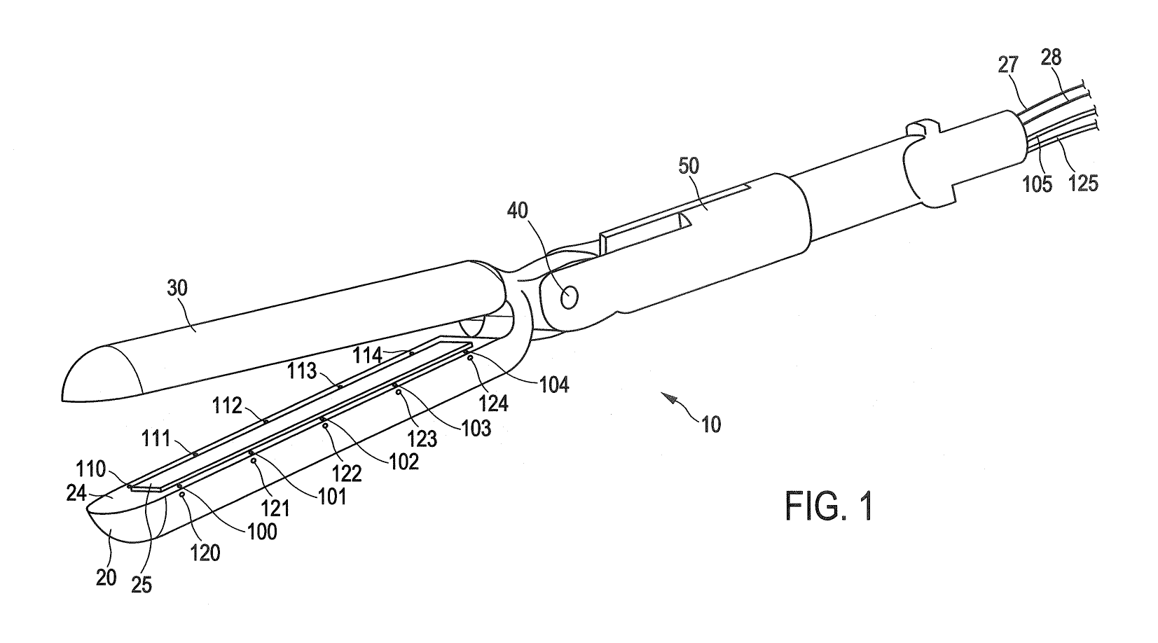

FIG. 1 shows a first embodiment of an electrosurgical instrument according to the first aspect of the invention.

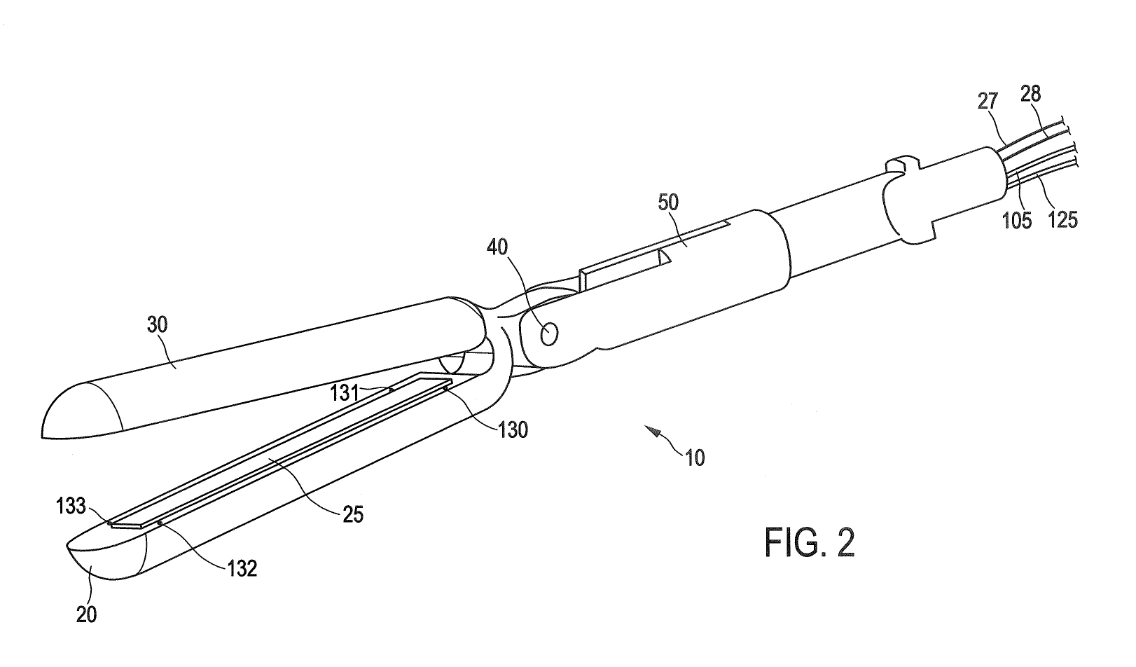

FIG. 2 shows a second embodiment of an electrosurgical instrument according to the first aspect of the invention.

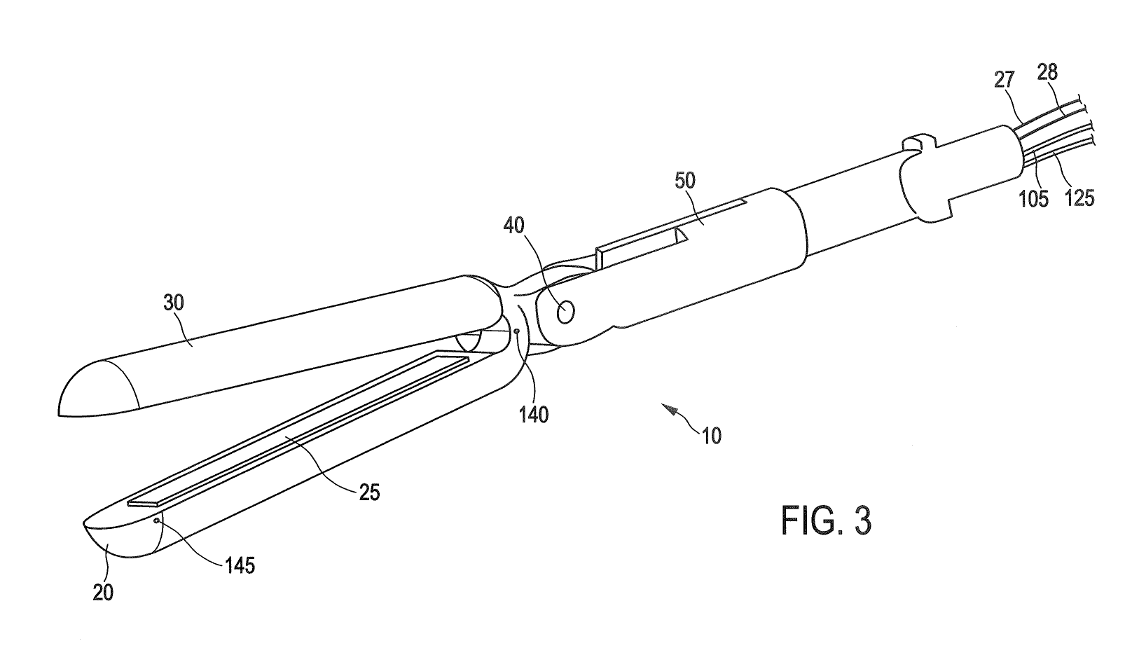

FIG. 3 shows a third embodiment of an electrosurgical instrument according to the first aspect of the invention.

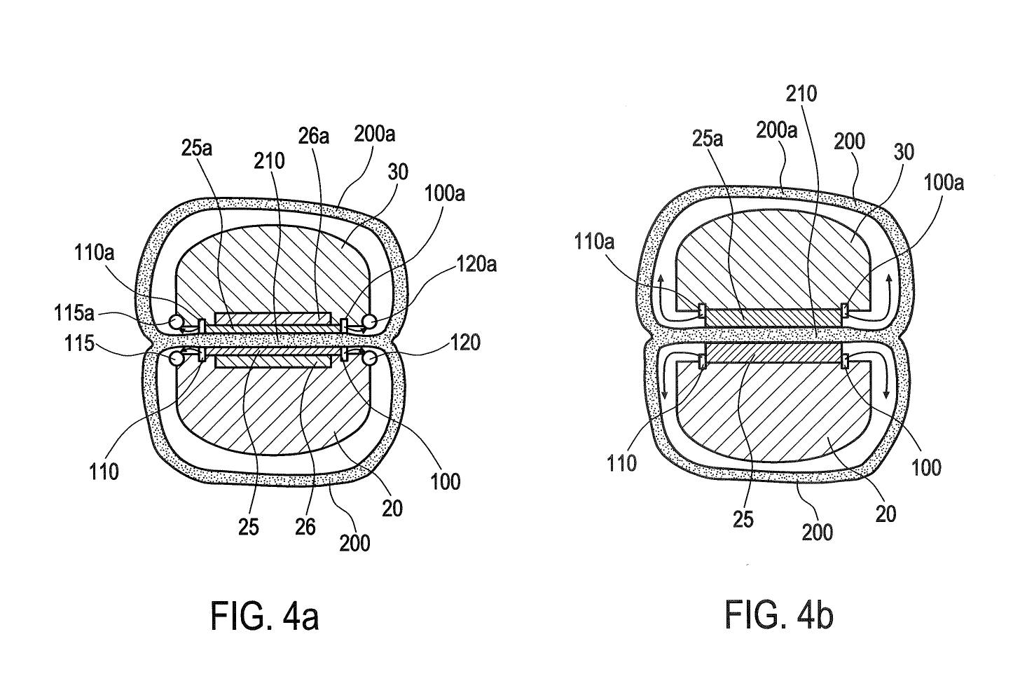

FIGS. 4a and 4b show schematically applications of electrosurgical instruments according to the first aspect of the invention.

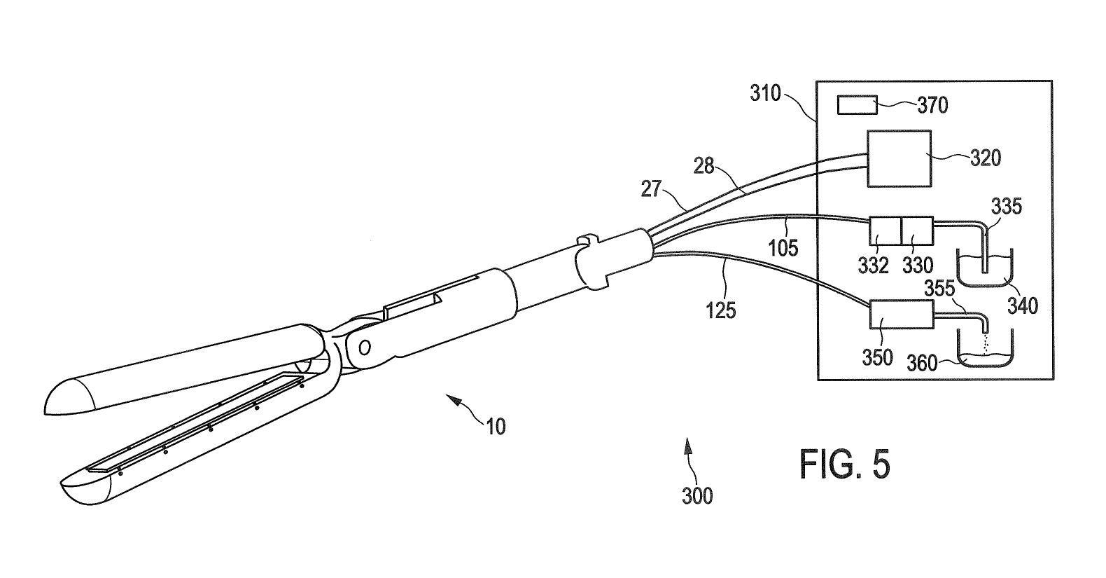

FIG. 5 shows an embodiment of an electrosurgical device according to the second aspect of the invention.

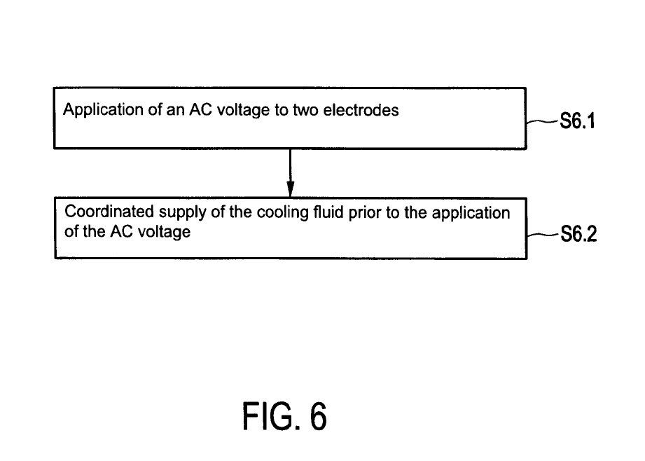

FIG. 6 shows a flow diagram of a method for operating an electrosurgical device according to a third aspect of the invention.

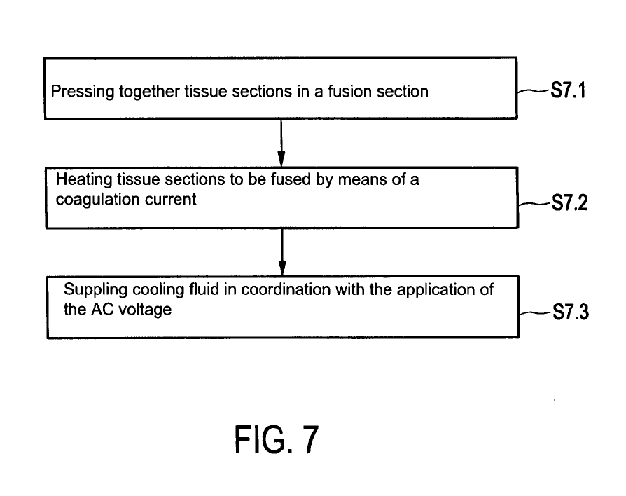

FIG. 7 shows a flow diagram of a method for tissue fusion according to the third aspect of the invention.

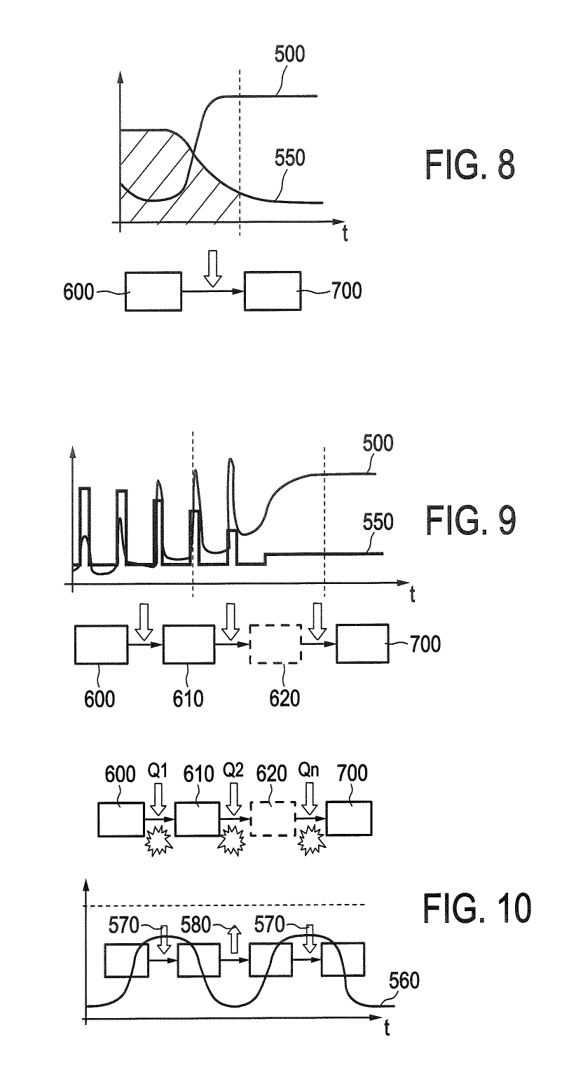

FIG. 8 shows the characteristics of energy supply and tissue resistance with continuous dehydration of tissue.

FIG. 9 shows the characteristics of energy supply and tissue resistance with dehydration of tissue through pulsed energy supply.

FIG. 10 shows the characteristics of the temperature with pulsed energy supply.

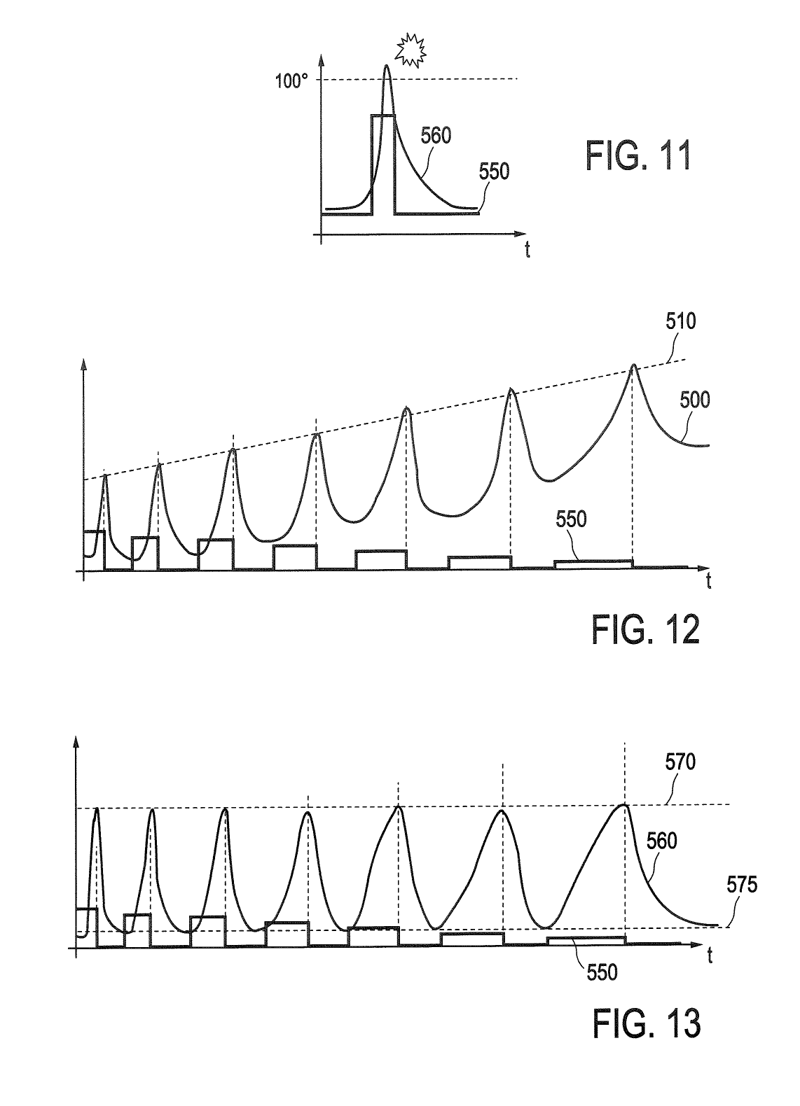

FIG. 11 shows a desired temperature characteristic in the tissue with application of a short HF pulse.

FIG. 12 shows the characteristics of supplied energy and tissue resistance with a resistance-controlled pulse/pause application.

FIG. 13 shows the characteristics of supplied energy and temperature with temperature-controlled pulse/pause application.

FIG. 1 shows a first embodiment of an electrosurgical instrument 10 according to the first aspect of the invention. The electrosurgical instrument 10 has a first branch 20 and a second branch 30. Both branches 20, 30 are pivotally connected to each other by means of a hinge 40, so that they can together perform a plier-like gripping movement. By means of the hinge 40, they are also connected to a handle part 50 of the electrosurgical instrument 10, at which the electrosurgical instrument can be supported or mounted.

On the first branch 20, an electrode 25 directing to the second branch 30 is arranged. The electrode 25 protrudes over a surrounding area 24 and thereby forms with its elevated surface a gripping surface. In order to facilitate the electrosurgical instrument 10 to be connected to a generator during operation, the electrode 25 is connected to a connecting wire circuit 27 which is led out of the electrosurgical instrument 10.

At the second branch 30, an electrode directing towards the first branch 20 is arranged, too, which is however not visible in the illustration according to FIG. 1. This additional electrode is connected to a connecting wire circuit 28, by which it can be also connected to a generator.

Laterally to the electrode 25, in the surrounding area 24, fluid outlets 100, 101, 102, 103, 104, 110, 111, 112, 113, 114 are arranged. The fluid outlets are presently arranged in two rows, wherein respectively one row is arranged along a longitudinal side of the electrode 25. By means of the fluid outlets 100, 101, 102, 103, 104, 110, 111, 112, 113, 114, a cooling fluid can be discharged laterally of the electrode.

The fluid outlets 100, 101, 102, 103, 104, 110, 111, 112, 113, 114 are connected to a fluid supply line 105, which is led out of the electrosurgical instrument 10. By means of the fluid supply line 105, the fluid outlets 100, 101, 102, 103, 104, 110, 111, 112, 113, 114 can be supplied with a cooling fluid, if e.g. the fluid supply line 105 is connected to a fluid pump. Such embodiment will be described with reference to FIG. 5.

Further, suction openings 120, 121, 122, 123, 124 are configured laterally at the first branch 20. For this purpose, at the opposite side not shown in FIG. 1, suction openings are also arranged in mirror symmetry, which are however not visible in this illustration. The suction openings 120, 121, 122, 123, 124 are connected to a fluid outlet channel 125. To this fluid outlet channel 125, e.g. a suction pump can be connected to, in order to provide for a negative pressure in the fluid outlet channel 125. Thereby, fluid discharging from the fluid outlets 100, 101, 102, 103, 104, 110, 111, 112, 113, 114 can be again withdrawn by suction. The in FIG. 1 not illustrated suction openings recited supra are also connected to the fluid outlet channel 125.

It is to be comprehended that the second branch 30 can be configured like the first branch 20. Such modification is shown in the application of FIG. 4a as will be described below.

FIG. 2 shows a second embodiment of an electrosurgical instrument 10 according to the first aspect of the invention. Components having the same function are designated with the same reference numerals like in FIG. 1 and are in the following not referred to again.

The electrosurgical instrument 10 of FIG. 2 differs from that in FIG. 1 such that, instead of the arrangement of fluid outlets and suction openings in respective rows, only a first fluid outlet 130 and a second fluid outlet 131 as well as a first suction opening 132 and a second suction opening 133 are provided. The fluid outlets 130, 131 are connected to the fluid supply line 105. Also the suction openings 132, 133 are connected to the fluid outlet 125.

The fluid outlets 130, 131 are provided at a longitudinal end of the electrode, i.e. here at that longitudinal end which is closer to the hinge 40, whilst the suction openings 132, 133 are arranged at the opposing longitudinal end of the electrode 25. By such arrangement, it can be achieved that a fluid stream extends along the longitudinal direction of the electrode and at both sides of the electrode. Thus, the fluid stream of the electrosurgical instrument 10 of FIG. 2 extends exactly transversally to the fluid stream of the electrosurgical instrument 10 of FIG. 1. Through the fluid stream extending along the longitudinal direction of the electrode, a to a large extent complete flushing of the electrode can be achieved, wherein resulting water vapor is particularly well absorbed by the cooling fluid.

FIG. 3 shows a third embodiment of an electrosurgical instrument 10 according to the first aspect of the invention. In contrast to the electrosurgical instruments shown in FIG. 1 and FIG. 2, this has a fluid outlet 140 which is arranged directly adjacent to the hinge 40. Thus, the fluid outlet 140 is not directly adjacent to the electrode, which can cause a broader fluid stream during operation.

A suction opening 145 is configured at a to the hinge 40 oppositely arranged end of the first branch 20. Thereby, a fluid stream can be guided at a larger distance and with a higher volume longitudinally along the electrosurgical instrument 10.

The fluid outlet 140 is connected to the fluid supply line 105 as well as the suction opening 145 is connected to the fluid outlet 125. It is to be comprehended that, also in the case of the electrosurgical instrument 10 of FIG. 3, on the side of the first branch 20 not illustrated in this figure, a fluid outlet and a suction opening are arranged mirror-symmetrically, which are not visible in FIG. 3.

FIG. 4a shows a possible application of an electrosurgical instrument 10 of FIG. 1. Thereby, the first branch 20 is introduced into a hollow tubular tissue section 200, e.g. intestine tissue, and the second branch 30 is also introduced into a hollow tubular tissue section 200a. The two tissue sections 200, 200a shall be fused along a tissue section 210.

In minor deviation of the embodiment of FIG. 1, in FIG. 4a an electrode 25a and fluid outlets 100a, 110a and suction openings 120a, 115a are shown not only at the first branch 20, but also at the second branch 40. Their arrangement and function is already directly apparent from the description of the embodiment of FIG. 1.

The tissue section 210 can be fused between the two electrodes 25, 25a. Simultaneously, between the illustrated fluid outlets 100, 110, 100 a and 110 a and the illustrated suction openings 120, 115, 120a, 115a, a fluid stream extending transversally to the longitudinal direction of the electrodes 25, 25 can be triggered. This fluid stream can directly neutralize a released water vapor, which is formed during fusing of tissue, by providing a heat sink in which the water vapor condenses, cools down and is withdrawn. Damage of tissue outside the area to be fused can thereby be prevented.

Further, the branches 20, 30 in FIG. 4a respectively comprise a heating unit 26, 26a which is arranged below the electrodes 25, 25a at the respective side facing away from the tissue.

FIG. 4b shows a minor deviation of the application of FIG. 4a. In deviation of FIG. 4a, no suction openings and no heating units are provided at the branches 20, 30. The cooling fluid discharging from the fluid outlets 100, 110, 110a is thus supplied to the surrounding of the electrosurgical instrument. There it can either accumulate or be removed by means of a separate hose.

FIG. 5 shows an embodiment of an electrosurgical device 300 according to a second aspect of the invention.

The electrosurgical device 300 comprises an electrosurgical instrument 10 as already described with reference to FIGS. 1 to 3. Therefore, in the following it is not further referred to in more detail to the electrosurgical instrument 10.

The electrosurgical device 300 further comprises a supply device 310. The supply device 310 comprises a generator 320, a fluid pump 330, a cooling unit 332, a fluid tank 340, a suction tube 335, a suction pump 350, an inlet tube 355 and a fluid waste container 360. The supply device 310 further comprises a controller 370 which can control the components of the supply device 310.

The HF generator 320 is connected to the electrodes of the electrosurgical instrument 10 by means of the connecting wire circuits 27, 28. Accordingly, the HF generator 320 can supply the electrodes with current and voltage in order to trigger an electrosurgical operation, like a fusion process.

The fluid pump 330 is connected to the suction tube 335 which is introduced into the fluid tank. Thereby, the fluid pump 330 can suck cooling fluid from the fluid tank 340. Further, the fluid pump 330 is connected to a cooling unit 332 which cools down the cooling fluid to a temperature of 1° C. to 3° C. The cooling unit 332 again is connected to the fluid supply line 105 of the electrosurgical instrument 10, wherein it is facilitated that the fluid pump 330 supplies a cooling fluid from the fluid tank 340 at the desired temperature to the fluid outlets (here not shown again) of the electrosurgical instrument 10.

The suction pump 350 is connected to the fluid outlet channel 125 of the electro-surgical instrument 10, wherein it can suck fluid from the suction openings of the electrosurgical instrument 10. For this purpose, the suction pump 350 creates a negative pressure in the fluid outlet channel 125. Further, the suction pump 350 is connected to an inlet hose 355, which opens out into the fluid waste container 360. Thereby, the suction pump 350 can guide the fluid suctioned from the electrosurgical instrument 10 into the fluid waste container 360, where it is stored in order to be disposed of later on.

The controller 370 can control the HF generator 320 and the fluid pump 330, the cooling unit 332 and the suction pump 350. According to the energy of the HF generator, the controller 370 will calculate which dose of cooling fluid is necessary to neutralize the water vapor which is formed during fusion such that a damage of the surrounding tissue is prevented. The controller 370 will control the fluid pump 330, the cooling unit 332 and the suction pump 350 accordingly.

The controller 370 controls the HF generator 320 such that it releases its energy in a pulsed manner. For this purpose, it uses the method of the pulse-pause-application which has already been described above.

The controller 370 can be integrated in the HF generator.

FIG. 6 shows a flow diagram of an embodiment of a method for operating an electrosurgical device according to the third aspect of the invention. Thereby, in step S 6.1, at first an AC voltage is applied to two electrodes of an electrosurgical device.

Then, in step 6.2, a cooling fluid is supplied, wherein the supply of the cooling fluid is coordinated with the AC voltage. This means that the cooling fluid is supplied in such dose and/or temperature that water vapor which is formed during fusion triggered by AC voltage can be neutralized as completely as possible so that it can no longer cause thermal damage of the surrounding tissue.

FIG. 7 shows a flow diagram of an embodiment of a method for tissue fusion according to a fourth aspect of the invention. Thereby, in step 7.1, at first tissue sections to be fused are pressed together. Subsequently, in step 7.2, the tissue sections are heated by means of a coagulation current. This is effected because the tissue is positioned between two electrodes of an electrosurgical instrument and an AC voltage, which induces the coagulation current, is applied to these electrodes. Additionally, the heating can also be effected by means of a heating unit.

Finally, in step 7.3, a cooling fluid is supplied in such manner that the supply is coordinated with the application of the AC voltage. This means that the cooling fluid is supplied in such dose and temperature that a water vapor which is formed during fusing is neutralized as complete as possible. Thereby, a damage of surrounding tissue is prevented.

FIG. 8 shows the characteristics of energy and tissue resistance with continuous dehydration of tissue, as it occurs if HF voltage is applied in continuous manner. Thereby, a constant RMS of the HF voltage is assumed. The horizontal axis of the illustrated diagram indicates the time and is therefore designated with t. The same applies for the following FIGS. 9 to 13.

The curve 500 shows the characteristics of the tissue resistance. It is apparent that it rises with increasing dehydration of the tissue. This is due to the fact that the electrical conductivity through tissue mainly takes place through electrolytic conduct which increasingly worsens with a decrease of the moisture content. Corresponding to the increasing resistance, the energy output illustrated in the curve 550 decreases. This is due to the known physical law that, at constant voltage, the energy output is inversely proportional to the resistance.

A tissue portion 600 in a condition before the treatment and a tissue portion 700 after the treatment are shown schematically. The tissue portion 700 after the treatment has a considerably lower moisture content compared to the tissue portion 600 before the treatment.

It is to be mentioned that the continuous application of HF voltage shown in FIG. 8 is not advantageous for numerous application.

FIG. 9 shows the characteristics of energy output and tissue resistance with pulsed application of HF voltage. Thereby, the tissue resistance is again illustrated by a curve 500, while the energy is illustrated by a curve 550.

As illustrated, the energy 550 is only supplied in short pulses. This takes place in that the respective HF voltage is supplied only within short pulses. The pulses have e.g. a length of 50 ms and the pauses between the pulses 500 ms. Due to the steep edge in the curve 550 of the energy output, the temperature of ebullition is reached quickly. When the temperature of ebullition is reached, however, the tissue resistance decreases very quickly due to the evaporating water. Hence, the output of a respective high energy is only possible for a short time. Otherwise, the danger of formation of electric arcs between the electrodes might occur which could destroy and carbonize the tissue.

As illustrated, the tissue resistance increases by each pulse. The energy output decreases pulse by pulse according to the context already referred to with respect to FIG. 8. This is due to the dehydration of the tissue, which is schematically shown in FIG. 9 on the basis of tissue sections 600, 610, 620, 700, the dehydration degree of which increases continuously. This is to be comprehended such that the tissue section 610 schematically illustrates the condition of the tissue section 600 after application of the first pulse. The tissue section designated with reference numeral 620 schematically shows a plurality of conditions which occur with continuously pulsed dehydration. The tissue section 700 then illustrates the final condition at maximum dehydration.

FIG. 10 shows the characteristics of the tissue temperature by means of a temperature curve 560 with pulsed vaporization and cooling. The individual conditions of schematically illustrated tissue sections 600, 610, 620, 700 are to be comprehended like those of FIG. 9, whereby in FIG. 10 additionally a respective application of energy is symbolized by arrow Q1, Q2, Qn and a respective vaporization of water is illustrated by a serrated symbol.

In the characteristics of the temperature, the correlation with application of energy and cooling is shown. A respective application of energy is illustrated by arrows 570, while a cooling, i.e. a decrease of energy, is illustrated by an arrow 580. The respective processes continue accordingly.

As it is apparent, the temperature rises during application of energy 570, in other words, it rises during application of a HF voltage within a pulse. In the pauses between the pulses, the temperature decreases, because due to cooling energy is dissipated.

FIG. 11 shows the characteristics of energy output illustrated in a curve 550 and the corresponding characteristics of the temperature illustrated in a curve 560 when a pulse is applied. At the beginning of the pulse, the temperature rises steeply and exceeds the temperature of ebullition of 100° C. Due to the subsequently evaporating water and the thereby decreasing resistance, the temperature already decreases before the end of the pulse in order to again decrease significantly after the end of the pulse. Thus, the temperature remains only for a short time above the temperature of ebullition, whereby also only a respective small part of the total water content evaporates per pulse. As already described, this facilitates the evacuation of the water vapor.

FIG. 12 shows the characteristics of the energy output illustrated in a curve 550 and the corresponding tissue resistance illustrated in a curve 500 during a resistance-controlled pulse/pause application. As shown, the energy is applied in individual pulses, wherein the voltage is maintained constant. Due to the already described effect tissue resistance which increases by each pulse, the absolute value of the energy output decreases continuously.

Bei einem Puls nimmt der Gewebewiderstand aufgrund des bereits beschriebe-nen Verdampfungseffekts deutlich zu. Der Puls dauert so lange, bis ein Schwellenwert 510 überschritten wird. Dann wird die HF-Spannung abgeschaltet and eine vordefinierte Zeit gewartet, bevor der nächste Puls aktiviert wird.

The threshold value 510 of the resistance is continuously elevated in order to take account of the increasing dehydration of the tissue. Thereby, pulse by pulse, respectively, higher threshold values are required which have to be reached before the pulse is terminated. Hence, also the length of the pulses is extended over the time.

FIG. 13 shows, in deviation of FIG. 12, the characteristics of energy output illustrated in a curve 550, and the corresponding characteristics of temperature illustrated in a curve 560 with a temperature-controlled pulse/pause application. Thereby, the HF voltage is always applied if a lower temperature 575 is underrun. Due to the then applied HF voltage, the temperature rises until it exceeds an upper temperature threshold 570. Then, the HF voltage is switched off again in order to allow the tissue to cool down.

As shown, the durations of pulses and pauses are thereby not fixed set points, but are determined dynamically during the application. This allows a particularly good adaptation of HF voltage to different kinds of tissue.

1. Electrosurgical instrument with a gripping surface and an electrode that is arranged at least in the area of the gripping surface, wherein outside of the gripping surface, adjacent to it, a fluid outlet is arranged which is connected to a fluid supply line for supplying a cooling fluid.

2. Electrosurgical instrument according to claim 1, comprising two gripping surfaces that are facing each other and movable towards each other.

3. Electrosurgical instrument according to claim 2, wherein the electrosurgical instrument has two branches being jointed with each other and being movable towards each other, and wherein the respective gripping surface is formed by a surface facing the other branch.

4. Electrosurgical instrument according to claim 1, wherein outside the gripping surface, adjacent to it, a suction opening for withdrawing the cooling fluid by suction is arranged.

5. Electrosurgical device comprising an electrosurgical instrument according to claim 1, and a fluid pump being connected to the fluid channel for supplying a cooling fluid, and a generator for generating a coagulation current being electrically connected to the in the area of the gripping surface arranged electrode of the electrosurgical instrument, wherein the fluid pump and the generator are connected to a controller which coordinates the operation of the fluid pump and the operation of the generator.

6. Electrosurgical device according to claim 5, wherein the controller is configured to control the generator such that it generates the coagulation current in a pulsed manner

7. Electrosurgical device according to claim 5, further comprising a suction pump by which the cooling fluid can be withdrawn by suction.

8. Electrosurgical device according to claim 7, wherein the electrosurgical instrument has outside the gripping surface, adjacent to it, a suction opening for withdrawing the cooling fluid by suction and that the suction pump is connected to the suction opening.

9. Electrosurgical device according to claim 5, wherein the fluid pump supplies during operation the cooling fluid at a temperature of 1°-3° C.

10. Method for operating an electrosurgical device, comprising the process steps:
applying an AC voltage to at least one electrode of a gripping surface of an electrosurgical instrument,
supplying a cooling fluid in direct proximity of the electrode in coordination with the application of AC voltage.

11. Method according to claim 10, wherein as cooling fluid a non-conductive fluid is used.

12. Method for tissue fusion, comprising the process steps:
pressing the tissue sections to be fused against each other in a fusion zone,
heating the tissue sections to be fused in the fusion zone,
cooling the tissue by supplying a cooling fluid adjacent to the fusion zone.

13. Method according to claim 12, wherein the step of heating comprises inducing coagulation current into the tissue sections to be fused.

14. Method according to claim 12, wherein the step of heating comprises a heating of tissue sections to be fused by means of a heating unit.

15. Method according to claim 10, which is performed by use of an electrosurgical device comprising:
an electrosurgical instrument with a gripping surface and an electrode that is arranged at least in the area of the gripping surface, wherein outside of the gripping surface, adjacent to it, a fluid outlet is arranged which is connected to a fluid supply line for supplying a cooling fluid; and
a fluid pump being connected to the fluid channel for supplying a cooling fluid, and a generator for generating a coagulation current being electrically connected to the in the area of the gripping surface arranged electrode of the electrosurgical instrument, wherein the fluid pump and the generator are connected to a controller which coordinates the operation of the fluid pump and the operation of the generator.
